(19) United States
(12) Patent Application Publication
(10) Pub. No.: US 2026/0294498 A1
(43) Pub. Date: Oct. 1, 2026

(54) LOCK INTERACTION FOR INTRAMEDULLARY NAIL AIMING ARM

(71) Applicant: DEPUY SYNTHES PRODUCTS, INC., Raynham, MA (US)

(72) Inventors: Jae STELZER, Norton, MA (US); Stanley J. KMIEC, Jr., Morgantown, PA (US); Kevin CLANCY, West Chester, PA (US); Tom KEYER, West Chester, PA (US); Evan STAMBLER, West Chester Township, OH (US)

(21) Appl. No.: 19/576,119

(22) Filed: Mar. 24, 2026

Related U.S. Application Data
(60) Provisional application No. 63/778,156, filed on Mar. 26, 2025.

(51) Int. Cl. A61B 17/88; A61B 17/00; A61B 17/56; A61B 17/72; A61B 17/90

(52) U.S. Cl. CPC A61B 17/8872; A61B 17/7291; A61B 17/90; A61B 2017/00367; A61B 2017/564

(57) ABSTRACT

An intramedullary nail system includes an insertion handle for insertion of an intramedullary nail into a medullary canal of a bone and an aiming arm to releasably couplable to the insertion handle. The arm includes a connecting hole and a plurality of openings extending therethrough, the connecting hole configured to slidably receive a distal portion of the insertion handle therethrough. A locking mechanism is configured to lock the arm in a desired orientation relative to the insertion handle and includes a pin movable between a locked configuration, in which a tip thereof extends into the connecting hole and one of the plurality of recesses of the insertion handle to lock the arm in the desired orientation relative to the insertion handle, and an unlocked configuration, in which the tip of the pin does not extend into the connecting hole so that the arm is rotatable thereabout.

### PRIORITY CLAIM

The present disclosure claims priority to U.S. Provisional Patent Application Ser. No. 63/778,156 filed Mar. 26, 2025; the disclosure of which is incorporated herewith by reference.

### BACKGROUND

Tibiotalocalcaneal (TTC) arthrodesis is a procedure involving fusion of the tibiotalar joint and the subtalar joint (talocalcaneal joint) to alleviate pain resulting from, for example, arthritic hindfoot or ankle joint, and/or to correct joint deformities. During a TTC fusion procedure, a nail is inserted into a medullary canal of a tibia via a plantar aspect of the foot so that the nail extends through the calcaneus, talus, and tibial bones. The nail may then be fixed relative to the bones via screws guided into the bone and through corresponding holes of the nail via, for example, an aiming arm that is rotatable about the nail to target a desired one of the corresponding holes. Upon rotation, the aiming arm is locked in position in alignment with the desired one of the corresponding holes before a screw is placed. Current systems may utilize a threaded knob or other mechanisms for locking the aiming arm relative to the nail. However, in some cases, this locking procedure may be time consuming and requires fine motor control.

### SUMMARY

The present disclosure relates to an intramedullary nail system. The nail system includes an insertion handle configured to facilitate insertion of an intramedullary nail into a medullary canal of a bone, the insertion handle extending from a distal end to a proximal end releasably couplable to the distal end of the intramedullary nail, the insertion handle including a plurality of recesses extending thereabout, each of the plurality of recesses extending along an exterior surface thereof. The nail system also includes an aiming arm configured to be releasably coupled to the insertion handle, the aiming arm including a connecting hole and a plurality of openings extending therethrough, the connecting hole being sized, shaped and configured to slidably receive a distal portion of the insertion handle therethrough. The aiming arm also includes a locking mechanism configured to lock the aiming arm in a desired orientation relative to the insertion handle in which one of the plurality of openings of the aiming arm is aligned with a corresponding hole of the intramedullary nail, the locking mechanism including a pin movable between a locked configuration, in which a tip thereof extends into the connecting hole to be received within one of the plurality of recesses of the insertion handle to lock the aiming arm in the desired orientation relative to the insertion handle, and an unlocked configuration, in which the tip of the pin does not extend into the connecting hole so that the aiming arm is rotatable thereabout, via an actuator.

In an embodiment, the aiming arm includes a connecting portion including the connecting hole, an aiming block including the plurality of openings extending therethrough, and a handle portion extending between the connecting portion and the aiming block, the pin of the locking mechanism housed within the handle portion.

In an embodiment, the pin extends longitudinally from a first end including the tip to a second end, the actuator configured to move the pin along a longitudinal axis thereof between the locked and unlocked configurations.

In an embodiment, the locking mechanism is biased toward the locked configuration.

In an embodiment, the actuator includes a push button extending from a distal surface of the handle portion.

In an embodiment, the actuator includes a wedged surface which interfaces with the second end of the pin which includes an angled surface so that, when the push button is pushed from a raised position toward a depressed position, the wedged surface slides along the angled surface of the second end of the pin to move the pin from the locked configuration toward the unlocked configuration.

In an embodiment, the actuator includes a slotted opening configured to engage a crossbar extending transversely through a portion of the pin, the slotted opening extending at an angle relative to the longitudinal axis of the pin so that when the push button is pushed from a raised position toward a depressed position, the crossbar slides along the slotted opening from a first end thereof toward a second end to move the pin from the locked configuration toward the unlocked configuration.

In an embodiment, the tip of the pin is tapered and corresponds in size and shape to the plurality of recesses of the insertion handle.

In an embodiment, the actuator includes a trigger rotatably connected to the second end of the pin so that moving the trigger between a first position and a second position relative to a handle portion of the aiming arm longitudinally translates the pin between the locked and unlocked configurations.

In an embodiment, the actuator includes a lever and a cammed portion which interfaces with the second end of the pin to move the pin longitudinally between the locked and unlocked configurations.

In an embodiment, the actuator includes a knob rotatably coupled to the second end of the pin so that rotation of the knob between a first position and a second position relative to a handle portion of the aiming arm moves the pin longitudinally between the locked configuration and the unlocked configuration.

In an embodiment, the actuator includes one of a push button, a lever, a trigger, and a knob, each of which is movable between a first position and a second position to move the pin between the locked configuration and the unlocked configuration.

The present disclosure also relates to an aiming device, comprising a connector portion including a connecting hole extending therethrough, the connecting hole being sized, shaped and configured to slidably receive a distal portion of an insertion handle of an intramedullary nail therethrough so that the aiming device is releasably coupleable with the insertion handle, and an aiming block including a plurality of openings extending therethrough, each of the plurality of openings extending therethrough along a central axis which, when the aiming block in a desired position relative to the insertion handle, aligns with a corresponding hole of the intramedullary nail. The aiming device also comprises a handle portion extending between the connector portion and the aiming block, the handle portion configured to be gripped via a user, and a locking mechanism including a pin housed within the handle portion and an actuator configured to move the pin between a locked configuration, in which a tip of the pin extends into the connecting hole, and an unlocked configuration, in which the tip does not extend into the connecting hole.

In an embodiment, the actuator includes one of a push button, a lever, a trigger, and a knob.

In an embodiment, the actuator includes a push button extending from a surface of the handle portion which, when the aiming device is in an operative configuration relative to the insertion handle, faces away from a bone, the actuator including an angled surface which slidably interfaces with an end of the pin to move the pin between the unlocked and locked configuration as the push button is pushed and released, respectively.

The present disclosure also relates to an exemplary method for treatment of bones. The method includes inserting an intramedullary nail through a medullary canal of a bone using an insertion handle coupled to an end thereof and releasably locking an aiming arm to a distal portion of the insertion handle in a first desired orientation relative thereto in which the distal portion is received within a connector opening of the aiming arm and one of a plurality of openings of the aiming arm is aligned with a first hole of the intramedullary nail, the aiming arm locked in the first desired orientation relative to the insertion handle via a locking mechanism of the aiming arm including a pin extending into the connector opening to engage a first one of a plurality of recesses along an exterior surface of the distal portion of the insertion handle received within the connector opening in a locked configuration. The method also includes inserting a first fixation element through the one of the plurality of openings and the first hole of the intramedullary nail, and releasing the aiming arm from the first desired orientation by moving an actuator from a first position to a second position, which correspondingly moves the pin from the locked configuration toward an unlocked configuration, in which the pin is removed from the first one of the plurality of recesses of the insertion handle and no longer extends into the connector opening so that the aiming arm is freely movable relative to the insertion handle.

In an embodiment, the method further includes rotating the aiming arm about the insertion handle to a second desired orientation relative to the insertion handle so that a second one of the plurality of openings of the aiming arm is aligned with a corresponding second hole of the intramedullary nail and locking the aiming arm in the second desired orientation relative to the insertion handle via the locking mechanism, and inserting a second fixation element through the one of the plurality of openings and the first hole of the intramedullary nail.

In an embodiment, releasing the aiming arm includes moving the actuator from the first position to the second position to correspondingly move the pin from the locked configuration toward the unlocked configuration.

In an embodiment, the actuator includes a push button such that pressing the push button moves the pin from the locked configuration toward the unlocked configuration.

In an embodiment, the locking mechanism is biased toward the locked configuration so that the pin is configured to revert toward the biased locked configuration as it is moved over one of the plurality of recesses of the insertion handle.

### BRIEF DESCRIPTION

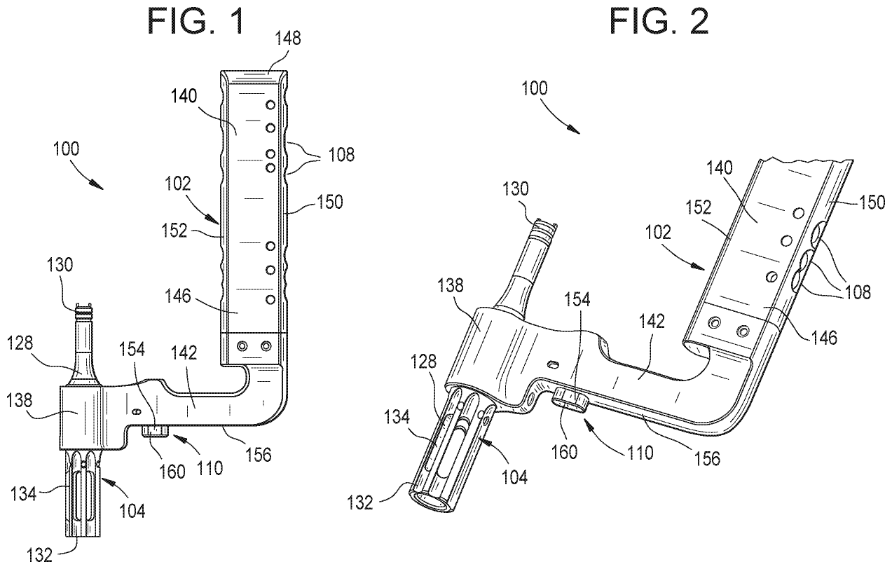

FIG. 1 shows a side view of an intramedullary nailing system according to an exemplary embodiment of the present disclosure.

FIG. 2 shows a perspective view of the exemplary system according to FIG. 1.

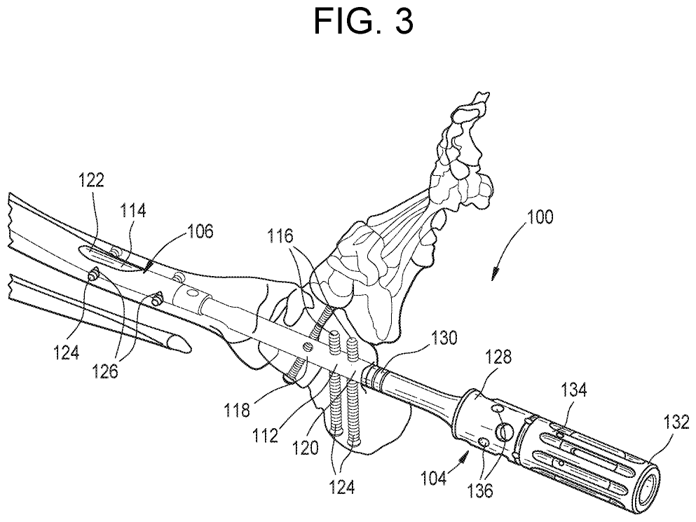

FIG. 3 shows a perspective view of an insertion device and an intramedullary nail according to the exemplary system of FIG. 1.

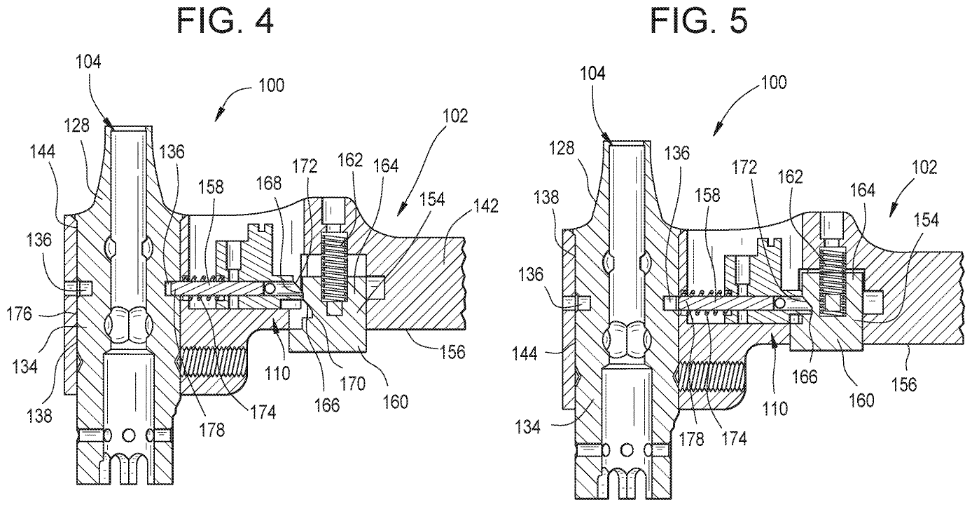

FIG. 4 shows a cross-sectional side view of the exemplary system according to FIG. 1, a locking mechanism of an aiming arm thereof in a locked configuration.

FIG. 5 shows a cross-sectional side view of the exemplary system according to FIG. 1, the locking mechanism of the aiming arm thereof in an unlocked configuration.

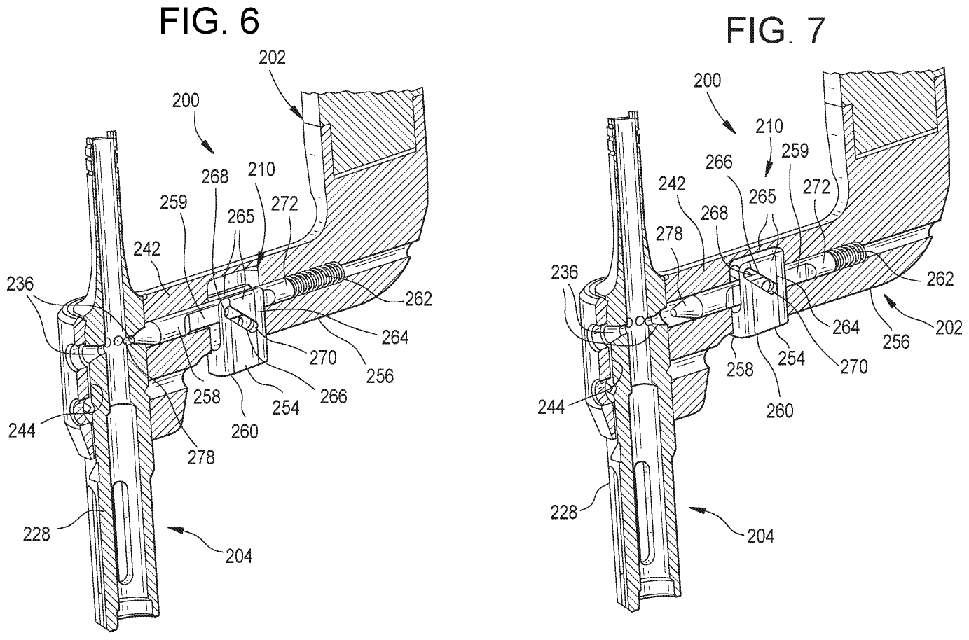

FIG. 6 shows a cross-sectional perspective view of a system according to another exemplary embodiment of the present disclosure, a locking mechanism if an aiming arm thereof in a locked configuration.

FIG. 7 shows a cross-sectional perspective view of the exemplary system according to the system of FIG. 6, the locking mechanism of the aiming arm thereof in an unlocked configuration.

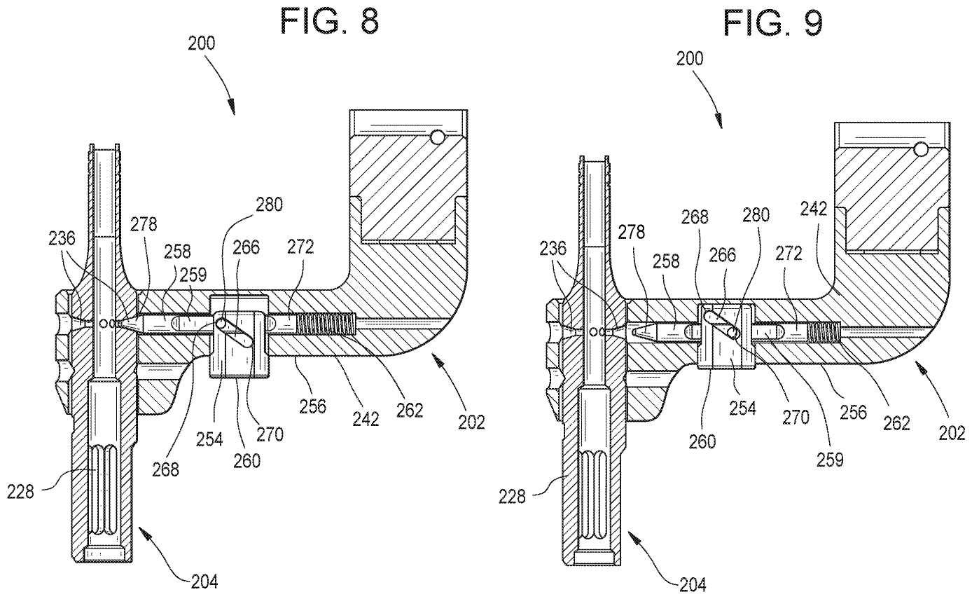

FIG. 8 shows a cross-sectional side view of the exemplary system according to FIG. 6 (in the locked configuration).

FIG. 9 shows a cross-sectional side view of the exemplary system according to FIG. 7 (in the unlocked configuration).

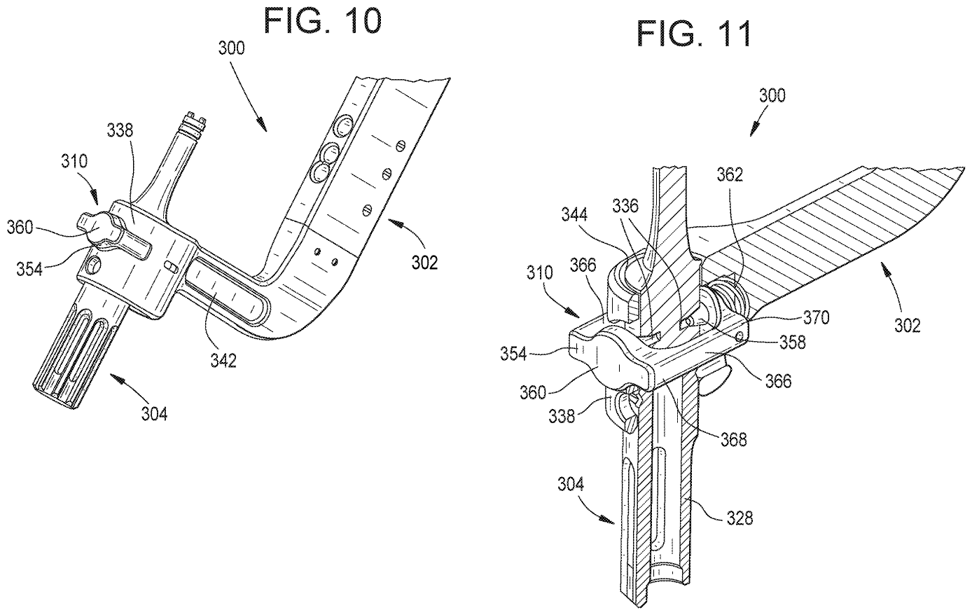

FIG. 10 shows a perspective view of a system according to another exemplary embodiment of the present disclosure.

FIG. 11 shows a partial cross-sectional perspective view of the exemplary system according to FIG. 10.

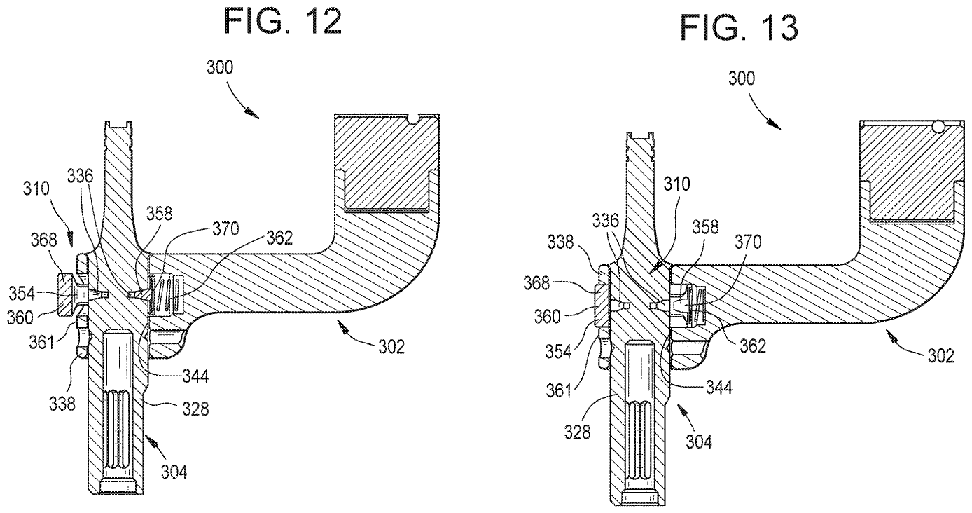

FIG. 12 shows a cross-sectional side view of the exemplary system according to FIG. 10, a locking mechanism of an aiming arm of the system in a locked configuration.

FIG. 13 shows a cross-sectional side view of the exemplary system according to FIG. 10, the locking mechanism in the unlocked configuration.

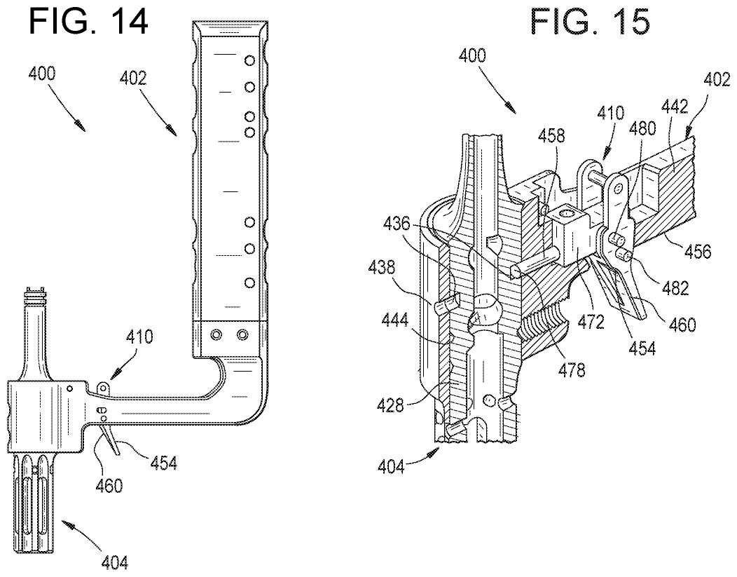

FIG. 14 shows a side view of a system according to yet another exemplary embodiment of the present disclosure.

FIG. 15 shows a cross-sectional perspective view of the exemplary system according to FIG. 14.

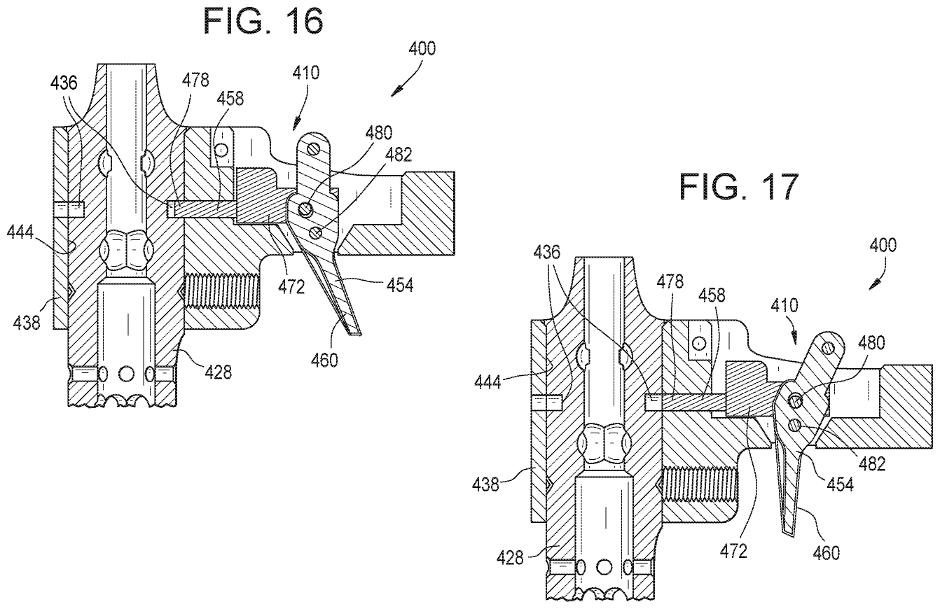

FIG. 16 shows a cross-sectional side view of the exemplary system according to FIG. 14, a locking mechanism of an aiming arm of the system in a locked configuration.

FIG. 17 shows a cross-sectional side view of the exemplary system according to FIG. 14, the locking mechanism in the unlocked configuration.

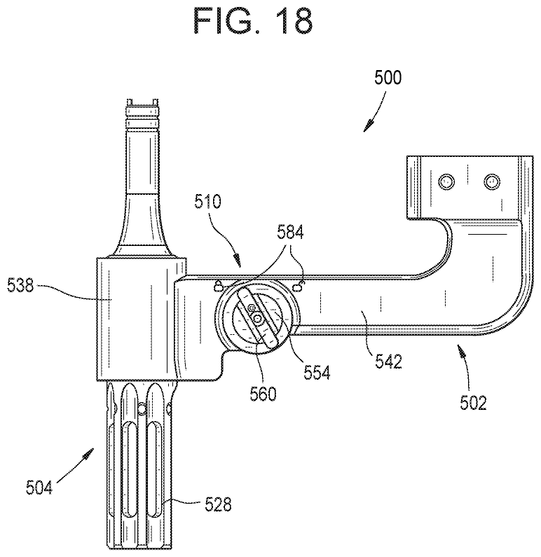

FIG. 18 shows a side view of a portion of a system according to another exemplary embodiment of the present disclosure.

FIG. 19 shows a partially transparent perspective view of a portion of the exemplary system according to FIG. 18, a locking mechanism of an aiming arm of the exemplary system in a locked configuration.

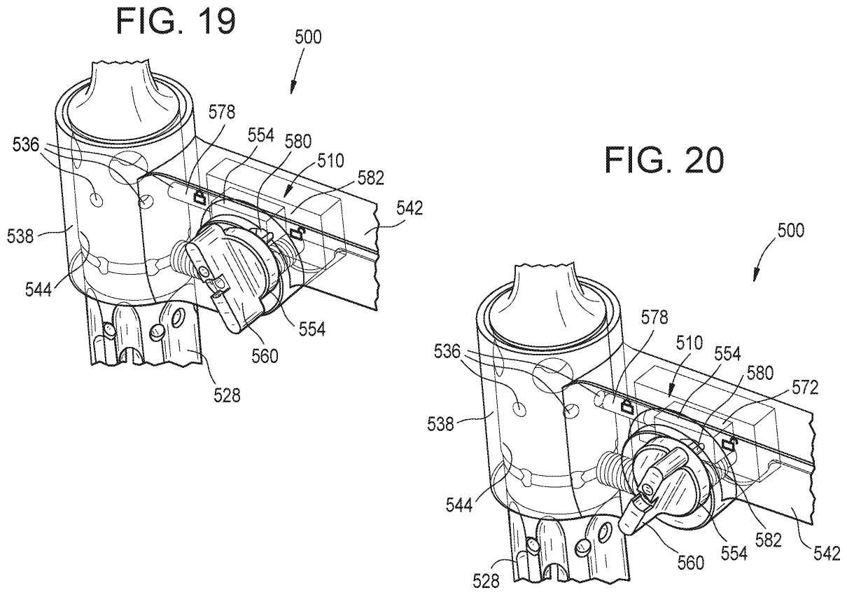

FIG. 20 shows another partially transparent perspective view of a portion of the exemplary system according to FIG. 18, the locking mechanism in the unlocked configuration.

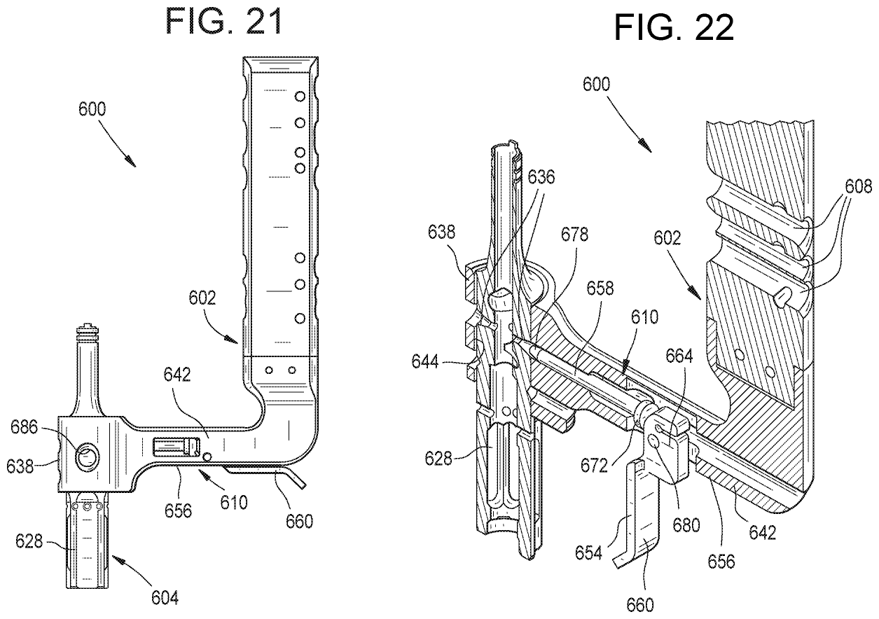

FIG. 21 shows a side view of a system according to yet another exemplary embodiment of the present disclosure.

FIG. 22 shows a cross-sectional perspective view of a portion of the exemplary system according to FIG. 21.

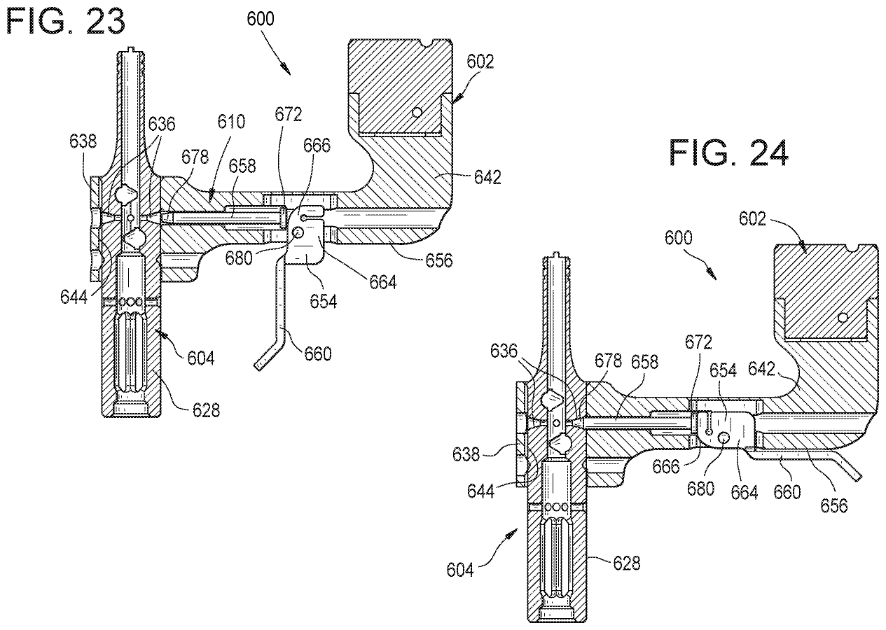

FIG. 23 shows a cross-sectional side view of a portion of the exemplary system according to FIG. 21, a locking mechanism of an aiming arm of the exemplary system in an unlocked configuration.

FIG. 24 shows another cross-sectional side view of a portion of the exemplary system according to FIG. 21, the locking mechanism of the exemplary system in a locked configuration.

### DETAILED DESCRIPTION

The present disclosure may be further understood with reference to the following description and appended drawings, wherein like elements are referred to with the same reference numerals. The present disclosure relates to an intramedullary nail system and, in particular, an aiming arm of an intramedullary nail system configured to be releasably coupled to an insertion device, via which an intramedullary (IM) nail is inserted into a medullary canal of a tibia during a TTC fusion procedure. Exemplary embodiments of the aiming arm include a locking mechanism configured to facilitate a quick lock and release of the aiming arm in a desired rotational position/orientation relative to the insertion device, and thereby the IM nail, in which one or more of openings of the aiming arm is aligned with one or more holes of the IM nail to facilitate a targeted insertion of a fixation element (e.g., locking screw) therethrough. In particular, the aiming arms according to these embodiments may be quickly and easily moved to a first desired position relative to the IM nail and locked in the first desired position.

One or more screws may be inserted through one or more holes of the IM nail via corresponding openings of the aiming arm, each of which guides a screw through the intramedullary nail at a desired angle relative thereto along an axis aligned with an axis of a selected locking hole in the IM nail. Upon placement of the one or more screws when the aiming arm is at the first desired position, the locking mechanism may be quickly released, rotated to a second desired rotational position/orientation relative to the IM nail, and locked in the second desired configuration to target a second group of holes which extend through the IM nail at a different angle and/or orientation relative to the bone than the holes through which screw(s) were inserted when the aiming arm was in the first desired position. This process of releasing, rotating, and locking the aiming arm relative to the insertion device may be repeated until fixation elements have been inserted through all of the holes of the IM nail which the user has determined should be utilized.

It will be understood by those of skill in the art that the terms “proximal” and “distal,” as used herein, correspond to an anatomical orientation of the exemplary system, in an operative position with proximal referring to a direction toward a trunk of the body while distal refers to a direction toward the extremity (e.g., with the hip being the proximal end of the leg and the foot being at the distal end of the leg). It will also be understood by those of skill in the art that although the exemplary embodiments are specifically shown and described with respect to a TTC fusion procedure, the system and method of the present disclosure may be utilized for the treatment of any of a variety of bones and/or joints in which an aiming arm may be used to guide screws through an implant such as, for example, an IM nail.

As shown in FIGS. 1-5, an intramedullary nailing system 100 according to an exemplary embodiment of the present disclosure comprises an aiming arm 102 configured to be releasably coupled to an insertion device 104, which is configured to facilitate insertion of an IM nail 106 into a medullary canal of a bone (e.g., tibia). The aiming arm 102 is configured to be rotated relative to the insertion device 104 (and the IM nail 106) and locked relative to the insertion device 104 in any of a number of locking positions/orientations, where each of the locking positions/orientations aligns one (or more) of a plurality of openings 108 of the aiming arm 102 with a corresponding hole or holes of the IM nail 106. The aiming arm 102 is configured to be locked relative to the insertion device 104 via a spring-loaded locking mechanism 110 so that the locking mechanism 110 may be quickly moved between an unlocked configuration in which the aiming arm 102 is rotatable relative to the insertion device 104, and a locked configuration in which the aiming arm 102 is rotationally locked relative to the insertion device 104 in one of the locking positions.

As shown in FIG. 3, the IM nail 106 extends along a longitudinal axis L from a distal end 120 to a proximal end 122. According to an exemplary embodiment, a proximal portion 114 of the IM nail 106 is sized, shaped and configured to be inserted into a medullary canal of a tibia via a plantar aspect of the foot so that, in an operative position, a distal portion 112 is received within the calcaneus and talus while the proximal portion 114 is received within the tibia. In an exemplary embodiment, the distal portion 112 is configured to releasably engage a portion of the insertion device 104. In some embodiments, the distal portion 112 may have a diameter larger than a diameter of the proximal portion 114. The distal portion 112, however, is not required to have a larger diameter than that of the proximal portion 114. For example, the distal portion 112 may have a diameter of 13 mm while the diameter of the proximal portion 114 may range between 9 mm and 13 mm. It will be understood by those of skill in the art, however, that these dimensions are exemplary only and that the IM nail 106 may have any of a variety of sizes, shapes and configurations as determined by the patient anatomy, etc.

A plurality of distal locking holes 116 extend through the distal portion 112 of the IM nail 106 with each of the holes 116 extending along a respective central axis extending transversely through the distal portion 112—i.e., the central axes of the holes 116 are angled with respect to the longitudinal axis L of the IM nail 106. In an exemplary embodiment, the central axis of each of the distal locking holes 116 is substantially perpendicular to the longitudinal axis L of the IM nail 106. In an exemplary embodiment, two of the distal locking holes 116 extend through the distal portion 112 equally distanced from the distal end 120 with central axes extending through the IM nail 106 substantially perpendicular to one another so that a user of the system 100 (e.g., surgeon) may insert a locking element 124 (e.g., locking screw) through a desired one of the distal locking holes 116, in a desired direction (e.g., lateral-medial or posterior-anterior). As described above, the distal locking holes 116 are positioned along the distal portion 112 of the IM nail 106 and configured so that, when the IM nail 106 is in an operative position, the locking elements 124 may be inserted through one or more of the distal locking holes 116 to fix the IM nail 106 relative to the calcaneus. In an exemplary embodiment, the locking elements 124 are configured to extend through the distal locking holes 116 to fuse a cuboid bone to the calcaneus.

In an exemplary embodiment, the distal portion 112 also includes a talus locking hole 118 extending through the IM nail 106 along a central axis transverse to the longitudinal axis L of the IM nail 106. The talus locking hole 118 of this embodiment extends through the distal portion 112 proximally of the distal locking holes 116. In an exemplary embodiment, the central axis of the talus locking hole 118 extends through the distal portion 112 at a non-perpendicular angle relative to the longitudinal axis L of the IM nail 106. An angle of the central axis of the talus locking hole 118 may be selected so that, when the IM nail 106 is in the operative position, the locking element 124 inserted through the talus locking hole 118 extends through the talus bone in a desired configuration (e.g., lateral-posterior to medial-anterior). Thus, a user may insert the locking element 124 through the talus locking hole 118 to fix the IM nail 106 relative to the talus. In an exemplary embodiment, the locking element 124 may be configured to extend through the talus locking hole 118 to fuse a navicular bone to the talus.

The above description of the distal locking holes 116 and the talus locking hole 118 are illustrative and should not be viewed as limiting. It will be understood by those of skill in the art that the distal portion 112 of the IM nail 106 may have any number of locking holes extending therethrough in any of a variety of configurations so long as the IM nail 106 is configured to be fixed relative to the calcaneus and/or talus, as desired. In another exemplary embodiment, the distal portion 112 includes a locking hole extending transversely therethrough along a central axis configured such that a fixation element inserted therealong extends across the subtalar joint—e.g., from the calcaneus, across the subtalar joint, and through the talus. It will be understood by those of skill in the art that the locking elements 124 may include any of a variety of fixation elements configured to be inserted through the distal locking holes 116 and the talus locking hole 118 to fix the IM nail 106 relative to bone.

The proximal portion 114 may also include one or more proximal locking holes 126 each extending along a respective central axis transverse to the longitudinal axis L of the IM nail 106. The proximal locking holes 126 are configured to receive the locking elements 124 therethrough along the central axes thereof to, when the IM nail 106 is in the operative position, fix the proximal portion 114 relative to the tibia. In an exemplary embodiment, the proximal locking holes are configured to receive the locking elements 124 therein in a medial-lateral direction. A position of each of the proximal locking holes 126 along the proximal portion 114 may vary depending on, for example, a length of the IM nail 106.

As will be understood by those of skill in the art, the IM nail 106 may have any of a variety of configurations so long as the IM nail 106 is configured to be inserted into a medullary canal of a bone to fix portions of the bone and or adjacent bones relative to one another. It will be understood by those of skill in the art that although the IM nail 106 is shown and described as having a specific configuration of holes (e.g., the proximal and distal locking holes 126, 116 and the talus locking hole 118), the IM nail 106 may include any of a variety of holes and/or openings in any of a variety of configurations where the holes and/or openings are configured to receive any of a variety of types of fixation elements.

In an exemplary embodiment, a proximal portion of the IM nail 106 is configured (sized, shaped, etc.) to be inserted into the medullary canal of the tibia using the insertion device 104, which includes an insertion handle 128 configured to facilitate insertion of the IM nail 106 to a desired position within the medullary canal. The insertion handle 128 is configured to be releasably engaged with the distal end 120 of the IM nail 106, in longitudinal alignment therewith. According to an exemplary embodiment, the insertion handle 128 extends longitudinally from a proximal end 130 configured to engage the distal end 120 of the IM nail 106, to a distal end 132. A distal portion 134 of the insertion handle 128 is configured to releasably engage the aiming arm 102 and includes a plurality of recesses 136 configured to receive a corresponding portion of the locking mechanism 110 therein, as will be described in further detail below. Each of the recesses 136 is positioned about the distal portion 134 so that, when engaged with the locking mechanism 110 of the aiming arm 102, locks the aiming arm 102 in a desired one of the locking positions relative to the IM nail 106—e.g., so that one of the openings 108 of the aiming arm 102 is aligned with a target one of the holes (e.g., distal locking holes 116, talus locking hole 118 or proximal locking holes 126) of the IM nail 106 through which it is desired to insert the locking element 124.

According to an exemplary embodiment, as shown in FIGS. 1-2 and 4-5, the aiming arm 102 includes a connecting portion 138 configured to releasably couple the aiming arm 102 to the insertion handle 128, an aiming block 140 including a plurality of openings 108 extending therethrough each being configured to guide the locking element 124 into a targeted one of the locking holes (116, 118, 126) of the IM nail 106, and a handle portion 142 extending between the connecting portion 138 and the aiming block 140.

The connecting portion 138 includes a connecting hole 144 extending therethrough. The connecting hole 144 is configured to slidably receive the distal portion 134 of the insertion handle 128 therein and extends through the connecting portion 138 along an axis aligned so that, when the connecting portion 138 is connected to the insertion handle 128, the axis of the connecting hole 144 is parallel to and/or coaxial with the longitudinal axis L of the IM nail 106. The connecting hole 144 is also configured so that the connecting portion 138, and thereby the aiming arm 102, is rotatable about the distal portion 134 of the insertion handle 128 received therein.

As shown in FIGS. 1-2, the aiming block 140 extends longitudinally from a distal end 146 to a proximal end 148 and includes the plurality of openings 108 extending transversely therethrough. The handle portion 142 extends from the connecting portion to the distal end 146 of the aiming block 140 so that, when the aiming arm 102 is coupled to the insertion device 104, the aiming block 140 extends substantially parallel to the longitudinal axis L of the IM nail 106. Each of the openings 108 extends through the aiming block 140 from a first surface 150 which, when the aiming arm 102 is in an operative configuration connected to the insertion device 104 faces away from the bone, to a second surface 152 which, when the aiming arm 102 in the operative configuration, faces toward the bone.

Each of the openings 108 extends through the aiming block 140 along a central axis which, when the aiming arm 102 is in one of the locking positions relative to the IM nail 106, is aligned with the central axis of a corresponding one of the locking holes of the IM nail (e.g., one of the distal locking holes 116, the talus locking hole 118, or the proximal locking holes 126). As described above, the aiming arm 102 is rotatable about the insertion handle 128 so that the aiming arm 102 is rotatable relative to the insertion device 104 between the locking positions, each of the possible positions aligning one or more of the openings 108 with a corresponding one of the locking holes (116, 118, 126) of the IM nail 106.

As described above, the handle portion 142 connects the connecting portion 138 to the distal end 146 of the aiming block 140. The handle portion 142 is also sized, shaped and configured to be gripped by the user of the system 100 so that the aiming arm 102 may be rotated relative to the insertion device 104 via gripping of the handle portion 142. The locking mechanism 110 is housed within the handle portion 142 and includes an actuator 154 including a push button 160 extending from a distal surface 156 of the handle portion 142 (i.e., a surface of the handle portion 142 facing away from the bone) to facilitate ergonomic handling thereof. In an exemplary embodiment, the push button 160 is positioned along the handle portion 142 such that, when the handle portion 142 is gripped by the user, the push button 160 is accessible for pressing via a thumb of the user.

According to an exemplary embodiment, as shown in FIGS. 4-5, the locking mechanism 110 includes a pin 158 movable between a locked configuration (see FIG. 4), in which a tip 178 of the pin 158 extends into the connecting hole 144 to engage one of the recesses 136 of the insertion handle 128, and an unlocked configuration (see FIG. 5), in which the tip 178 does not extend into the connecting hole 144 so that the connecting portion 138, and thereby the aiming arm 102, is free to rotate about the insertion handle 128. According to an exemplary embodiment, the pin 158 extends longitudinally from the tip 178 at a first end to a second end 172 and is movable along a longitudinal axis thereof between the locked and the unlocked configurations.

The tip 178 of the pin 158 is sized, shaped and configured to correspond to a configuration (e.g., size, shape) of the recesses 136 so that when the pin 158 is received therein, the aiming arm 102 is locked relative thereto-i.e., the aiming arm 102 cannot move or rotate relative to the IM nail 106. The pin 158 is movable between the locked and unlocked configuration via the actuator 154. As described above, the actuator 154 may be configured as a push button 160 extending distally from the distal surface 156 of the handle portion 142 of the aiming arm 102. The push button 160 of this embodiment is biased toward a raised configuration via a first biasing element 162 (e.g., spring). An internal portion 164 of the actuator 154 includes an angled surface 166 tapering from a first end 168 toward a second end 170 to act as a wedge that interfaces with an angled surface at the second end 172 of the pin 158 to move the pin 158 between the locked and unlocked configurations.

As described above, the pin 158 extends longitudinally from the second end 172, which interfaces with the actuator 154, to the tip 178. The actuator 154 moves the pin 158 longitudinally between the locked and unlocked configurations. In an exemplary embodiment, the pin 158 is biased toward the actuator 154 via a second biasing element 174 (e.g., spring) so that the pin 158 maintains engagement/interface with the actuator 154 at all times—e.g., as the locking mechanism 110 is moved between the locked and unlocked configuration. In particular, in the locked configuration, the actuator 154 is biased toward the raised configuration (via the first biasing element 162) so that the angled surface of the second end 172 of the pin 158 engages the first end 168 (e.g., a wide portion of the wedge) of the angled surface 166 of the actuator 154. Thus, the pin 158 is biased toward the locked configuration.

When, however, the push button 160 is pushed toward the distal surface 156, the angled surface of the second end 172 of the pin 158 maintains engagement with the actuator 154 and is slid along the angled surface 166 of the actuator 154 toward the second end 170 of the angled surface 166 (e.g., tapered portion of the wedge) so that the pin 158 is moved toward the unlocked configuration in which the tip 178 does not extend into the connecting hole 144. Thus, during initial assembly of the aiming arm 102 with the insertion device 104 and/or when it is desired to move the aiming arm 102 from a first rotational position/orientation to a second rotational position/orientation relative to the insertion device 104, the user presses the push button 160 so that the lock mechanism 110 is in the unlocked configuration and the connecting portion 138 is free to move about and along the distal portion 134 of the insertion handle 128.

According to an exemplary method utilizing the system 100, the IM nail 106 is inserted into a medullary canal of a tibia through a plantar aspect of a foot using the insertion device 104. In particular, the insertion handle 128 is coupled to the IM nail 106 and the IM nail 106 is inserted through the bones so that the distal portion 112 of the IM nail 106 extends through the talus and calcaneus while the proximal portion 114 of the IM nail 106 extends through the tibia, as shown in FIG. 3. Upon insertion of the IM nail 106 into the medullary canal of the tibia, the aiming arm 102 is coupled to the distal portion 134 of the insertion handle 128 in a first rotational position/orientation relative the insertion handle 128.

As described above, the aiming arm 102 of this embodiment is coupled to the insertion handle 128 by moving the locking mechanism 110 from the locked configuration to the unlocked configuration (e.g., by pressing the push button 160) so that the connector portion 138 of the aiming arm 102 may be slid over the distal portion 134 of the insertion handle 128. According to an exemplary embodiment, the connecting portion 138 of the aiming arm 102 is slid over the distal end 132 of the insertion handle 128 of the insertion device 104 so that the distal portion 134 is received within the connecting hole 144 thereof.

In some embodiments, the user may continue to maintain the push button 160 in the depressed configuration—e.g., toward the distal surface 156 of the handle portion 142) until the aiming arm 102 is seated in a first desired rotational position/orientation relative to the insertion handle 128. Upon release of the push button 160, the actuator 154 reverts toward the biased locked configuration, pushing the pin 158 into the recess 136 of the insertion handle 128, which corresponds to the desired rotations position/orientation. In another embodiment, after an initial depression of the push button 160 to move the locking mechanism 110 from the locked configuration to the unlocked configuration, the user releases the push button 160 so that the pin 158 is maintained in the unlocked configuration by contact between the pin 158 and an exterior surface 176 of the distal portion 134. The user continues to rotate the aiming arm 102 relative to the insertion handle 128 until the pin 158 is moved over the recess 136 of the insertion handle 128 which corresponds to the desired first rotations position/orientation. As the pin 158 is moved over the recess 136, the actuator 154 is permitted to revert toward its biased configuration so that the pin 158 clicks or snaps into place within the recess 136.

In an exemplary embodiment, in the first rotational/position orientation, the central axis of one (or more) of the openings 108 of aiming arm 102 is aligned with the central axis of one or more of the distal locking holes 116. Thus, in the first rotation position/orientation, the locking element 124 may be inserted into the corresponding hole 116(s) via the opening of the aiming arm 102. Although not shown or described, it will be understood by those of skill in the art that the locking element 124 may be inserted through one or more of the distal locking holes 116 via the use of one or more of a protection sleeve, a guide sleeve, a guidewire, drills, etc.

Upon insertion of the locking element 124 through one or more of the distal locking holes 116, the aiming arm 102 may be moved from the first locking position/orientation relative to the insertion handle 128 to a second locking position/orientation relative to the insertion handle 128 by pushing the push button 160 so that the aiming arm 102 is rotatable relative to the insertion device 104. The aiming arm 102 is then rotated until the pin 158 is received within a recess 136 of the insertion handle 128 corresponding to the second locking position/orientation. According to one example, in the second rotational position/orientation, one of the openings 108 of the aiming arm 102 is aligned with, for example, the talus locking hole 118 of the IM nail 106. Thus, in the second rotation position/orientation, the locking element 124 may be inserted into the talus locking hole 118 via the opening of the aiming arm 102.

After insertion of the locking element 124 through talus locking hole 118, the aiming arm 102 may be moved from the second locking position/orientation relative to the insertion handle 128 to a third locking position/orientation relative to the insertion handle 128 by pushing the push button 160 so that the aiming arm 102 is rotatable relative to the insertion device 104. The aiming arm 102 may then be rotated until the pin 158 is locked within a recess 136 of the insertion handle 128 corresponding to the third locking position/orientation upon release of the push button 160. According to one example, in the third locking position/orientation, one of the openings 108 of the aiming arm 102 may be aligned with, for example, one or more of the proximal locking holes 126 of the IM nail 106. Thus, in the third locking position/orientation, the locking element 124 may be inserted into the proximal locking hole 126 via the opening of the aiming arm 102.

Although the exemplary method describes three locking positions/orientations of the aiming arm 102 relative to the insertion handle 128, it will be understood by those of skill in the art that the aiming arm 102 may be configured to be positioned in any number of possible positions/orientations relative to the insertion handle 128 so long as one or more of the openings 108 of the aiming arm 102 is in alignment with one or more of the holes of the IM nail 106. It will also be understood by those of skill in the art that the aiming arm 102 may be moved between the various possible positions/orientations, as desired, to target insertion of the locking elements 124 through holes of the IM nail 106 in any desired sequence.

Once all of the locking elements 124 have been placed (e.g., inserted through the IM nail 106) as desired, the aiming arm 102 may be removed from insertion handle 128 by once again pressing the push button 160 of the actuator 154 to move the locking mechanism 110 from the locked configuration to the unlocked configuration so the pin 158 is withdrawn from the connecting hole 144 (and the recess 136) to release the insertion handle 128 therefrom.

According to another exemplary embodiment, as shown in FIG. 6-9, an intramedullary nail system 200 according to another exemplary embodiment similarly comprises an aiming arm 202 configured to be releasably coupled to an insertion device 204 for inserting an IM nail into a medullary canal of a bone. The aiming arm 202 and the insertion device 204 may be substantially similar to the aiming arm 102 and insertion device 104, as described above with respect to the system 100.

In particular, the aiming arm 202 is configured to be rotated relative to the insertion device 204 and locked relative thereto in any one of a number of discrete locking positions/orientations, each of which aligns one or more of a plurality of openings of the aiming arm 202 with corresponding holes of the IM nail—e.g., distal locking holes, proximal locking holes, or a talus locking hole. Similarly to the aiming arm 102, the aiming arm 202 is configured to be locked relative to the insertion device 204 via a spring-loaded locking mechanism so that the locking mechanism 210 may be quickly and easily moved between an unlocked configuration, in which the aiming arm 202 is rotatable relative to the insertion device 104, and a locked configuration, in which the aiming arm 102 is locked in any one of the number of locking positions/orientations relative to the insertion device 104.

Similarly to the locking mechanism 110, the locking mechanism 210 includes an actuator 254 configured to move a pin 258 between the locked configuration (see FIGS. 6 and 8), in which the pin 258 extends into a connector opening 244 of the aiming arm 202 to be received within a recess 236 of an insertion handle 228 of the insertion device 204, and the unlocked configuration (see FIGS. 7 and 9), in which the pin 258 does not extend into the connector opening 244 so that the aiming arm 202 is free to move (e.g., rotate) relative to the insertion handle 228. Rather than an angled surface forming a wedge, however, the actuator 254 in this embodiment includes a slotted opening 266 engaging a crossbar 280 extending through the pin 258. As will be described in further detail below, the slotted opening 266 is angled with respect to a longitudinal axis of the pin 258 so that when a push button 260 of the actuator is depressed toward a distal surface 256 of the handle portion 242 of the aiming arm 202, the pin 258 is moved from the locked configuration toward the unlocked configuration.

In an exemplary embodiment, the pin 258 is movably housed within the handle portion 242 of the aiming arm 202 and extends longitudinally from a first end 272 to a second end 278 sized, shaped, and configured to be received in any one of the recesses 236 of the insertion handle 228. The pin 258 is housed within the handle portion 242 so that the pin 258 is movable along a longitudinal axis thereof between the locked configuration and the unlocked configuration. The second end 278 may include a tapered tip to facilitate receipt within a correspondingly sized and shaped recess 236 of the insertion handle 228.

In an exemplary embodiment, a biasing element 262 such as, for example, a spring, is positioned adjacent the first end 272 to bias the pin 258 toward the locked configuration. The pin 258 may also include flat portions 259 extending along a portion of a length of the pin 258, along opposing surfaces thereof. In an exemplary embodiment, the crossbar 280 extends transversely through a portion of the pin 258, substantially perpendicular to the longitudinal axis thereof. In an exemplary embodiment, the crossbar 280 may extend through the flat portions 259 such that the crossbar 280 extends perpendicular to the flat portions 259. As will be described in further detail below, the crossbar 280 is configured to engage the actuator 254.

Similarly to the actuator 154, the actuator 254 includes a push button 260 extending distally from a distal surface 256 of the handle portion 242 of the aiming arm 202 (e.g., from a surface of the aiming arm facing away from the foot). According to an exemplary embodiment, an internal portion 264 of the actuator 24 that is received within the handle portion 242 includes a pair of wings 265 with each of the wings 265 including the slotted opening 226 so that the pair of wings 265 are mirror images of one another. The wings 265 extend along opposing surfaces of the pin 258 to receive a portion of the crossbar 280 therein. In an exemplary embodiment, the wings 265 extend over the flat portions 259 so that the wings 265 are slidable along the flat portions 259 as the pin 258 is moved between the locked and unlocked configurations.

When the pin 258 is in the biased locked configuration, the crossbar 280 is received within a first end 268 of the slotted opening 266. The slotted opening 266 is angled with respect to the longitudinal axis of the pin 258. Thus, as the push button 260 is pressed toward the distal surface 256, the crossbar 280 slides along the slotted opening 266 from the first end 268 toward a second end 270 compressing the biasing element 262 and moving the pin 258 toward the unlocked configuration. Thus, similarly to the locking mechanism 110 described above with respect to the system 100, depressing the push button 260 frees the aiming arm 202 for movement (e.g., rotation) relative to the insertion device 204 and releasing the push button 260 permits the locking mechanism to revert toward the biased locked configuration so that the aiming arm 202 is locked in a desired locking position/orientation relative to the insertion device 204. It will be understood by those of skill in the art that the system 200 may be utilized in a manner substantially similar to the system 100.

A system 300 according to another exemplary embodiment is shown in FIGS. 10-13. Except as described below, the system 300 is substantially similar to the system 200 described above, comprising an aiming arm 302 configured to be assembled with and releasably locked relative to an insertion device 304, in any of a number of possible rotational positions/orientations relative thereto, via a locking mechanism 310. The locking mechanism 310 may be substantially similar to the locking mechanism 210. Rather than an actuator including a push button along a distal surface of a handle portion 342 of the aiming arm 302, however, an actuator 354 of the locking mechanism 310 includes a push button 360 along a connecting portion 338 of the aiming arm 302, as shown in FIG. 10.

Similarly to the locking mechanism 210, the locking mechanism 310 may be moved between a locked configuration (see FIG. 12), in which a pin 358 thereof extends into a connector opening 344 to engage one of a plurality of recesses 336 of an insertion handle 328 of the insertion device 304, and an unlocked configuration (see FIG. 13), in which the pin 358 does not extend into the connector opening 344 so that the aiming arm 302 is free to rotate relative to the insertion handle 328. As will be described in further detail below, however, in this embodiment, the pin 358 is integrally formed with the actuator 354.

According to an exemplary embodiment, as shown in FIGS. 11-13, the actuator 354 extends longitudinally from a first end 368 including the push button 360 to a second end 370 including the pin 358. The actuator 354 further includes a pair of struts 366 connecting the push button 360 to the pin 358. The actuator 354 is housed within the aiming arm 302 so that the struts 366 extend through a wall of the connecting portion 338 along opposing sides of the connector opening 344 so as not to interfere therewith. In an exemplary embodiment, the push button 360 extends from an exterior surface 361 of the connecting portion 338, on a side of the connecting portion 338 opposing the handle portion 342. The pin 358 extends from the second end 370 of the actuator 354 in a direction extending toward the connector opening 344.

In an exemplary embodiment, the locking mechanism 310 further comprises a biasing element 362 such as, for example, a spring, which biases the actuator 354, and thereby the pin 358, toward the locked configuration, in which the pin 358 extends into the connector opening 344 to engage one of the recesses 336 of the insertion handle 328. A user may push the push button 360 toward the exterior surface 361 to move the actuator 354 and the pin 358 toward the unlocked configuration—e.g., to remove the pin 358 from the recess 336. As described above with respect to the systems 100, the aiming arm 302 may be rotated or otherwise movable relative to the insertion handle 328 when the locking mechanism 310 is in the unlocked configuration.

As shown in FIGS. 14-17, a system 400 according to yet another embodiment is substantially similar to the system 100 described above except as described below. The system 400 includes an aiming arm 402 configured to be releasably locked to an insertion device 404 in any of a number of possible rotational positions/orientations relative thereto, via a locking mechanism 410. Similarly to the locking mechanism 110, the locking mechanism 410 includes a pin 458 movable between a locked configuration (see FIG. 16), in which a tip 478 of the pin 458 extends into a connector opening 444 of the aiming arm 402 to engage one of a plurality of recesses 436 of an insertion handle 428 of the insertion device 404 received therein, and an unlocked configuration (see FIG. 17), in which the tip 478 does not extend into the connector opening 444 so that the aiming arm 402 is free to move/rotate about the insertion handle 428 received therein, via an actuator 454. The actuator 454 in this embodiment, however, is configured as a trigger 460.

Similarly to the pin 158, the pin 458 is housed within a handle portion 442 of the aiming arm 402 and extends longitudinally from a first end including the tip 478 to a second end 472 so that the pin 458 is movable between the locked and the unlocked configuration via a longitudinal movement thereof. According to an exemplary embodiment, the actuator 454 is rotationally connected to the second end 472 of the pin 458 at a first point 480 and rotationally connected to the handle portion 442 at a second point 482. In an exemplary embodiment, the trigger 460 extends from a distal surface 456 of the handle portion 442.

In an exemplary embodiment, similarly to the systems described above, the pin 458 may be biased toward the locked configuration. Thus, as the trigger 460 is pushed in a direction toward the connector portion 438, the actuator 454 pivots about the second point 482, resulting in a longitudinal translation of the pin 458 from the locked configuration toward the unlocked configuration as the actuator 454 rotates relative to the pin 458 via the first point 480. It will be understood by those of skill in the art that the aiming arm 402 including the locking mechanism 410 may be utilized in a manner substantially similarly to the system 100 described above.

A system 500 according to another embodiment is shown in FIGS. 18-20. The system 500 is substantially similar to the system 400 described above except as specifically pointed out below. The system 500 includes an aiming arm 502 configured to be releasably locked to an insertion device 504 in any of a number of a discrete number of locking positions/orientations relative thereto, via a locking mechanism 510. Similarly to the locking mechanism 410, the locking mechanism 510 includes a pin 558 movable between a locked configuration (see FIG. 19), in which a tip 578 of the pin 558 extends into a connector opening 544 of the aiming arm 502 to engage one of a plurality of recesses 536 of an insertion handle 528 of the insertion device 504 received therein, and an unlocked configuration (see FIG. 20), in which the tip 578 does not extend into the connector opening 544 so that the aiming arm 502 is free to move/rotate about the insertion handle 528 received therein, via an actuator 554. The actuator 554 in this embodiment, however, is configured as a rotatable knob 560 configured to move the pin 558 between the locked and unlocked configurations.

The pin 558 of this embodiment is substantially similar to the pin 458, extending from the tip 578 at a first end thereof to a second end 572, the pin 558 housed within a handle portion 542 and movable between the locked and unlocked configurations via a longitudinal movement thereof. Similarly to the actuator 454, the actuator 554 is rotationally connected to the second end 572 of the pin 558 at a first point 580 and to the handle portion 542 at a second point 582. The actuator 554 is configured as a knob 560, however, that is movable between a first position, in which the pin 458 is in the locked configuration, and a second position, in which the pin 458 is in the unlocked configuration. For example, the knob 560 may be rotated in a first direction about the second point 582 to move the pin 558 toward the locked configuration. In particular, as the knob 560 is rotated about the second point 582, the pin 558 rotates relative to the actuator 554 about the first point 580, which translates into a longitudinal movement of the pin 558. The knob 560 may be rotated in a second direction opposite the first direction to move the pin 558 longitudinally toward the unlocked configuration.

According to an exemplary embodiment, the pin 558 is not biased toward the locked configuration. Rather, the knob 560 is movable between the first and second positions to move the pin 558 between the locked and unlocked configurations, respectively, as desired. The handle portion 542 may include optional markings 584 thereon indicating a position of the knob 560 in the locked and unlocked configurations. It will be understood by those of skill in the art that the system 500 may be used in a manner substantially similar to the systems described above.

A system 600 according to another embodiment is shown in FIGS. 21-24. The system 600 is substantially similar to the system 500 described above except as pointed out below. The system 600 comprises an aiming arm 602 configured to be releasably locked to an insertion device 604 in any of a number of discrete locking positions/orientations relative thereto, via a locking mechanism 610. Similarly to the locking mechanism 610, the locking mechanism 610 includes a pin 658 movable between a locked configuration, in which a tip 678 of the pin 658 extends into a connector opening 644 of the aiming arm 602 to engage one of a plurality of recesses 636 of an insertion handle 628 of the insertion device 604 received therein, and an unlocked configuration, in which the tip 678 does not extend into the connector opening 644 so that the aiming arm 602 is free to move/rotate about the insertion handle 628 received therein, via an actuator 654. Similarly to the actuator 554, the actuator 654 is movable between a first position, in which the pin 658 is in the unlocked configuration (see FIG. 23), and a second position (see FIG. 23), in which the pin 658 is in the locked configuration. The actuator 654 of this embodiment, however, is configured as a lever 660 movable relative to a hand portion 642 of the aiming arm 602 to move the pin 658 between the locked and unlocked configurations.

Similarly to the pin 558, the pin 658 is housed within the handle portion 642 and extends longitudinally from a first end including the tip 678 to a second end 672 so that the pin 458 is movable between the locked and the unlocked configuration via a longitudinal movement thereof. Rather than being rotationally connected to the actuator 654, however, the second end 672 engages a cam portion 664 of the actuator 654. In an exemplary embodiment, similarly to the pin 158, the pin 658 is biased toward the actuator 654 so that the second end 672 maintains an engagement with the cam portion 664 at all times—e.g., as the locking mechanism 610 is moved between the locked and unlocked configurations.

In an exemplary embodiment, the actuator 654 includes the cam portion 664, which is housed within the handle portion 642, and the lever 660 which extends out of the handle portion 642, from a distal surface 656 thereof. The lever 660 is movable between a first position in which the lever 660 extends transversely away from the distal surface 656 and a second position in which the lever 660 is moved toward the distal surface 656 so that the lever extend substantially along the distal surface 656. As will be described in further detail below, when the lever 660 is in the first position the pin 658 is in the unlocked configuration and when the lever 660 is in the second position the pin 658 is in the locked configuration.

The cam portion 664 may be rotationally fixed relative to the handle portion 642 at a point 680 so that as the lever 660 is moved between the first and second locking positions relative to the handle portion 642, actuator rotates about the point 680. The cam portion 664 includes a cam surface 666 that interfaces with the second end 672 of the pin 658. The cam portion 664 is sized, shaped and configured so that, when the lever 660 is in the first locking position, the pin 658 is positioned relative to the handle portion 642 so that the pin 658 does not extend into the connector opening 644. When, however, the lever 660 is moved from the first locking position toward the second locking position, the second end 672 of the pin 658 slides along the cam surface 666, which is shaped to longitudinally move the pin 658 toward the locked configuration such the tip 678 of the pin 658 extends into the connector opening 644 to engage one of the recesses 636 of the insertion handle 628. As will be understood by those of skill in the art, the lever 660 may be moved between the first and second positions to move the locking mechanism 610 between the unlocked and locked configurations, respectively, as desired.

In an exemplary embodiment, the connector portion 638 may further include a window 686 via which markings along the insertion handle 628 are visible to indicate to a user a position of the aiming arm 602 relative to the insertion device 604. For example, the markings may indicate to the user when one or more of the openings 608 of the aiming arm is aligned with a desired one or more holes of an IM nail coupled to the insertion device 604. Although the exemplary embodiment shows and describes a window for determining alignment of the aiming arm 602 relative to the insertion device 604, it will be understood by those of skill in the art that the aiming arm 602 may include any of a variety of features for determining a relative alignment of the aiming arm 602 and the insertion device 604.

In another embodiment, for example, the connector portion 638 may include markings configured to be aligned with markings along the insertion handle 628 to align one or more of the openings 608 of the aiming arm with a corresponding one or more holes of the IM nail. Thus, the markings may indicate a relative position of the aiming arm 602 and the insertion device 604. It will be understood by those of skill in the art that the system 600 may be utilized in a manner substantially similar to the systems described above.

It will be appreciated by those skilled in the art that changes may be made to the embodiments described above without departing from the inventive concept thereof. It should be further appreciated that structural features and methods associated with one of the embodiments can be incorporated into other embodiments. It is understood, therefore, that this invention is not limited to the particular embodiment disclosed, but rather, modifications are also covered within the scope of the present invention as defined by the appended claims.

What is claimed is:

1. An intramedullary nail system, comprising:
an insertion handle configured to facilitate insertion of an intramedullary nail into a medullary canal of a bone, the insertion handle extending from a distal end to a proximal end releasably couplable to the distal end of the intramedullary nail, the insertion handle including a plurality of recesses extending thereabout, each of the plurality of recesses extending along an exterior surface thereof; and
an aiming arm configured to be releasably coupled to the insertion handle, the aiming arm including a connecting hole and a plurality of openings extending therethrough, the connecting hole being sized, shaped and configured to slidably receive a distal portion of the insertion handle therethrough, the aiming arm including a locking mechanism configured to lock the aiming arm in a desired orientation relative to the insertion handle in which one of the plurality of openings of the aiming arm is aligned with a corresponding hole of the intramedullary nail, the locking mechanism including a pin movable between a locked configuration, in which a tip thereof extends into the connecting hole to be received within one of the plurality of recesses of the insertion handle to lock the aiming arm in the desired orientation relative to the insertion handle, and an unlocked configuration, in which the tip of the pin does not extend into the connecting hole so that the aiming arm is rotatable thereabout, via an actuator.

2. The system of claim 1, wherein the aiming arm includes a connecting portion including the connecting hole, an aiming block including the plurality of openings extending therethrough, and a handle portion extending between the connecting portion and the aiming block, the pin of the locking mechanism housed within the handle portion.

3. The system of claim 2, wherein the pin extends longitudinally from a first end including the tip to a second end, the actuator configured to move the pin along a longitudinal axis thereof between the locked and unlocked configurations.

4. The system of claim 1, wherein the locking mechanism is biased toward the locked configuration.

5. The system of claim 3, wherein the actuator includes a push button extending from a distal surface of the handle portion.

6. The system of claim 5, wherein the actuator includes a wedged surface which interfaces with the second end of the pin which includes an angled surface so that, when the push button is pushed from a raised position toward a depressed position, the wedged surface slides along the angled surface of the second end of the pin to move the pin from the locked configuration toward the unlocked configuration.

7. The system of claim 5, wherein the actuator includes a slotted opening configured to engage a crossbar extending transversely through a portion of the pin, the slotted opening extending at an angle relative to the longitudinal axis of the pin so that when the push button is pushed from a raised position toward a depressed position, the crossbar slides along the slotted opening from a first end thereof toward a second end to move the pin from the locked configuration toward the unlocked configuration.

8. The system of claim 1, wherein the tip of the pin is tapered and corresponds in size and shape to the plurality of recesses of the insertion handle.

9. The system of claim 3, wherein the actuator includes a trigger rotatably connected to the second end of the pin so that moving the trigger between a first position and a second position relative to the handle portion of the aiming arm longitudinally translates the pin between the locked and unlocked configurations.

10. The system of claim 3, wherein the actuator includes a lever and a cammed portion which interfaces with the second end of the pin to move the pin longitudinally between the locked and unlocked configurations.

11. The system of claim 3, wherein the actuator includes a knob rotatably coupled to the second end of the pin so that rotation of the knob between a first position and a second position relative to the handle portion of the aiming arm moves the pin longitudinally between the locked configuration and the unlocked configuration.

12. The system of claim 1, wherein the actuator includes one of a push button, a lever, a trigger, and a knob, each of which is movable between a first position and a second position to move the pin between the locked configuration and the unlocked configuration.

13. An aiming device, comprising:
a connector portion including a connecting hole extending therethrough, the connecting hole being sized, shaped and configured to slidably receive a distal portion of an insertion handle of an intramedullary nail therethrough so that the aiming device is releasably coupleable with the insertion handle;
an aiming block including a plurality of openings extending therethrough, each of the plurality of openings extending therethrough along a central axis which, when the aiming block in a desired position relative to the insertion handle, aligns with a corresponding hole of the intramedullary nail;
a handle portion extending between the connector portion and the aiming block, the handle portion configured to be gripped via a user; and
a locking mechanism including a pin housed within the handle portion and an actuator configured to move the pin between a locked configuration, in which a tip of the pin extends into the connecting hole, and an unlocked configuration, in which the tip does not extend into the connecting hole.

14. The device of claim 13, wherein the actuator includes one of a push button, a lever, a trigger, and a knob.

15. The device of claim 13, wherein the actuator includes a push button extending from a surface of the handle portion which, when the aiming device is in an operative configuration relative to the insertion handle, faces away from a bone, the actuator including an angled surface which slidably interfaces with an end of the pin to move the pin between the unlocked and locked configuration as the push button is pushed and released, respectively.

16. A method for treatment of bones, comprising:
inserting an intramedullary nail through a medullary canal of a bone using an insertion handle coupled to an end thereof;
releasably locking an aiming arm to a distal portion of the insertion handle in a first desired orientation relative thereto in which the distal portion is received within a connector opening of the aiming arm and one of a plurality of openings of the aiming arm is aligned with a first hole of the intramedullary nail, the aiming arm locked in the first desired orientation relative to the insertion handle via a locking mechanism of the aiming arm including a pin extending into the connector opening to engage a first one of a plurality of recesses along an exterior surface of the distal portion of the insertion handle received within the connector opening in a locked configuration;
inserting a first fixation element through the one of the plurality of openings and the first hole of the intramedullary nail; and
releasing the aiming arm from the first desired orientation by moving an actuator from a first position to a second position, which correspondingly moves the pin from the locked configuration toward an unlocked configuration, in which the pin is removed from the first one of the plurality of recesses of the insertion handle and no longer extends into the connector opening so that the aiming arm is freely movable relative to the insertion handle.

17. The method of claim 16, further comprising:
rotating the aiming arm about the insertion handle to a second desired orientation relative to the insertion handle so that a second one of the plurality of openings of the aiming arm is aligned with a corresponding second hole of the intramedullary nail and locking the aiming arm in the second desired orientation relative to the insertion handle via the locking mechanism; and
inserting a second fixation element through the one of the plurality of openings and the first hole of the intramedullary nail.

18. The method of claim 16, wherein releasing the aiming arm includes moving the actuator from the first position to the second position to correspondingly move the pin from the locked configuration toward the unlocked configuration.

19. The method of claim 16, wherein the actuator includes a push button such that pressing the push button moves the pin from the locked configuration toward the unlocked configuration.

20. The method of claim 16, wherein the locking mechanism is biased toward the locked configuration so that the pin is configured to revert toward the biased locked configuration as it is moved over one of the plurality of recesses of the insertion handle.
